(19) United States
(12) Patent Application Publication
(10) Pub. No.: US 2025/0239270 A1
(43) Pub. Date: Jul. 24, 2025

(54) ELECTRONIC APPARATUS AND METHOD FOR CLASSIFYING COGNITIVE IMPAIRMENT BASED ON LARGE LANGUAGE MODEL

(71) Applicant: ELECTRONICS AND TELECOMMUNICATIONS RESEARCH INSTITUTE, Daejeon (KR)

(72) Inventors: Jeonguk BANG, Daejeon (KR); Byung Ok Kang, Daejeon (KR); SEUNG HOON HAN, Daejeon (KR)

(21) Appl. No.: 18/771,290

(22) Filed: Jul. 12, 2024

(30) Foreign Application Priority Data
Jan. 18, 2024 (KR) 10-2024-0008329

(51) Int. Cl. G10L 25/66; A61B 5/00; G10L 15/02; G10L 15/06; G10L 15/183; G10L 15/22; G10L 15/30

(52) U.S. Cl. CPC G10L 25/66; A61B 5/4088; A61B 5/4803; A61B 5/7264; G10L 15/02; G10L 15/063; G10L 15/183; G10L 15/22; G10L 2015/225; G10L 15/30

(57) ABSTRACT

Provided is an electronic apparatus including: an interface module configured to generate a first prompt related to a fluency evaluation request based on utterance voice of a user, and acquire evaluation feedback related to fluency based on the first prompt through a large language model; a first extraction unit configured to extract acoustic features based on the utterance voice; a second extraction unit configured to extract linguistic features based on transcribed text corresponding to the utterance voice and the evaluation feedback; and a classification module configured to classify a cognitive impairment group to which the user belongs based on the acoustic features and the linguistic features.

### CROSS-REFERENCE TO RELATED APPLICATION

This application claims priority to and the benefit of Korean Patent Application No. 10-2024-0008329, filed on Jan. 18, 2024, the disclosure of which is incorporated herein by reference in its entirety.

### BACKGROUND

### 1. Field of the Disclosure

Various embodiments disclosed in this disclosure relate to cognitive impairment test technology.

### 2. Description of Related Art

Patients with dementia—a chronic neurodegenerative disease that affects memory, thinking, cognitive abilities and the ability to perform simple tasks—are increasing in number. Dementia may be screened by a cognitive test, such as the Mini-Mental State Examination (MMSE) or the Montreal Cognitive Evaluation (MoCA), which uses a pen and paper. However, scoring of cognitive tests relies on the subjective judgment of clinical practitioners, which may lead to potential errors or differences in judgment between evaluators. To resolve these issues, various methods have been proposed to automatically predict whether dementia is present or not.

The related art of the automated dementia prediction method may be performed by acquiring voice of a user or transcribed text (e.g., text resulting from voice recognition), extracting acoustic features and linguistic features associated with dementia or mild cognitive impairment from the voice or the transcribed text, and determining whether cognitive intelligence is declining using a machine learning model trained on the various types of extracted feature information.

In related art of the automated dementia prediction methods, the various types of feature extracting is extensively studied. For example, the main acoustic features used include formants, pitch, phoneme duration, pause duration, a speech rate, shimmer, jitter, and mel-frequency cepstral coefficients (MMFCs). The linguistic features used include vocabulary size, word repetition, word specificity, word deprivation, interjection, sentence complexity, phrase length, and grammatical errors.

Recently, with advancements in artificial intelligence and machine learning technology, technologies utilizing embedding vectors extracted from a pre-trained model, such as Wav2Vec 2.0 and BERT, as acoustic and linguistic feature information are also being used.

### SUMMARY OF THE DISCLOSURE

However, the related art of automated dementia prediction methods require much time and effort to implement a separate learning model. For example, it is difficult to collect data on dementia or mild cognitive impairment, which makes it difficult to construct training data for artificial intelligence models.

Recently, there has been growing interest in artificial intelligence chatbot systems using general-purpose large language models. ChatGPT, which is a large language model trained through reinforcement learning with human feedback, may generate realistic and accurate responses to user queries. Utilizing such a large language model for predicting mild cognitive impairment or dementia allows a dementia prediction model to be constructed with a small amount of training data.

Various embodiments disclosed in this disclosure may provide an electronic apparatus and a method for classifying cognitive impairment based on a large language model capable of examining cognitive impairment based on evaluation feedback on user's utterance voice and features of the utterance voice.

According to an aspect of the present disclosure, there is provided a test apparatus including: an interface module configured to generate a first prompt related to a fluency evaluation request based on utterance voice of a user, and acquire evaluation feedback related to fluency based on the first prompt through a large language model; a first extraction unit configured to extract acoustic features based on the utterance voice; a second extraction unit configured to extract linguistic features based on transcribed text corresponding to the utterance voice and the evaluation feedback; and a classification module configured to classify a cognitive impairment group to which the user belongs based on the acoustic features and the linguistic features.

According to an aspect of the present disclosure, there is provided a method of classifying cognitive impairment, the method including: generating a first prompt related to a fluency evaluation request based on transcribed text corresponding to utterance voice of a user; acquiring evaluation feedback related to fluency in response to the first prompt through a large language model; extracting acoustic features and linguistic features based on the utterance voice, the transcribed text, and the evaluation feedback; and classifying a cognitive impairment group to which the user belongs based on the acoustic features and the linguistic features.

According to an aspect of the present disclosure, there is provided a test apparatus comprising: a memory in which at least one instruction related to an artificial intelligence model is stored; and a processor functionally connected to the memory, the processor executing the at least one instruction to: generate a first prompt related to a fluency evaluation request based on transcribed text corresponding to utterance voice of a user; input the first prompt to a large language model, and acquire evaluation feedback from the large language model; extract acoustic features and linguistic features based on the utterance voice, the evaluation feedback, and the transcribed text; and classify a cognitive impairment group to which the user belongs based on the acoustic features and the linguistic features.

Various embodiments disclosed in this disclosure may examine cognitive impairment based on evaluation feedback on user's utterance voice and features of the utterance voice.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a block diagram illustrating a configuration of a system for classifying cognitive impairment according to an embodiment.

FIG. 2 is a block diagram illustrating a configuration of a test apparatus according to an embodiment.

FIG. 3 is a block diagram illustrating a detailed configuration of a test apparatus according to an embodiment.

FIGS. 4 and 5 are examples of transcribed text generated by recording voice of users describing the “cookie theft” photo and transcribing the voice.

FIG. 6 is an example of a first prompt according to one embodiment.

FIGS. 7 and 8 are examples of fluency evaluation feedback based on a large language model for an utterance of a user in a dementia group and an utterance of a user in a normal group according to an embodiment.

FIG. 9 is an example of a test result according to one embodiment. and

FIG. 10 is a flowchart showing a method of classifying cognitive impairment according to one embodiment.

In relation to the description of the drawings, identical or similar reference numerals may be used for identical or similar components.

### DETAILED DESCRIPTION OF EXEMPLARY EMBODIMENTS

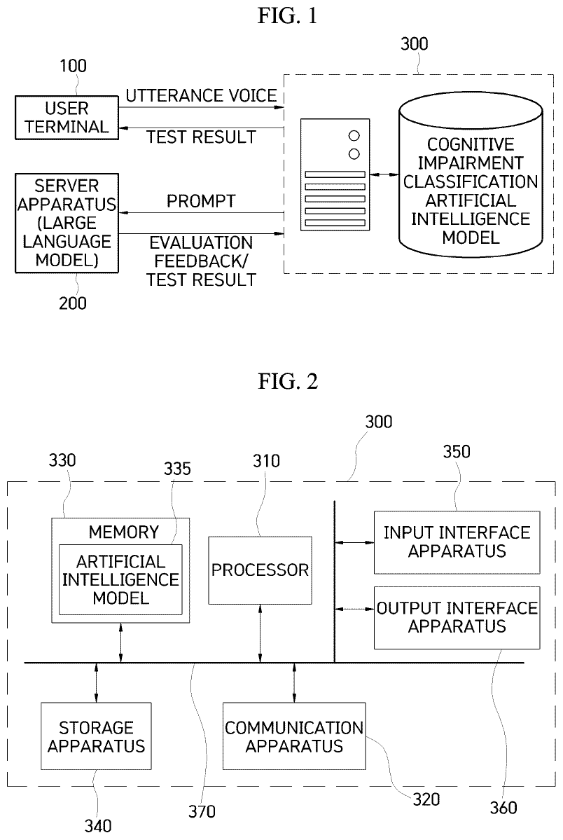

FIG. 1 is a block diagram illustrating a configuration of a system for classifying cognitive impairment according to an embodiment.

Referring to FIG. 1, a system 10 for classifying cognitive impairment according to an embodiment may include a user terminal 100, a server apparatus 200, and a test apparatus 300. In one embodiment, parts of the user terminal 100, the server apparatus 200, and the test apparatus 300 may be omitted or may be integrated. For example, the user terminal 100 and the test apparatus 300 may be configured as one apparatus.

According to one embodiment, the user terminal 100 may acquire a voice utterance of a user (e.g., an examinee) through a microphone. The utterance voice may include, for example, a user's utterance describing an image (e.g., a photo or a painting). The user terminal 100 may transmit the acquired utterance voice to the test apparatus 300 through a designated communication channel.

In one embodiment, the user terminal 100 may be a computing apparatus acquiring a utterance voice of a user and including a microphone, a display, a communication module, and a processor. The processor may detect or receive a utterance voice through the microphone and provide the utterance voice to the test apparatus 300 through a communication module (320 in FIG. 2). A processor 310 may acquire a test result from the test apparatus 300 through a communication channel and provide the test result to a user through the display.

According to one embodiment, the server apparatus 200 may include a large language model (e.g., ChatGPT). When the large language model acquires a prompt from the test apparatus 300 through a designated communication channel, the large language model may generate result text (e.g., evaluation feedback or test results) corresponding to the prompt. The large language model may provide the result text to the test apparatus 300 via a designated communication channel.

According to one embodiment, the test apparatus 300 may acquire utterance voice from the user terminal 100 through a designated communication channel. The test apparatus 300 may convert the utterance voice into text to generate transcribed text.

According to one embodiment, the test apparatus 300 may generate a request prompt related to a fluency evaluation based on the transcribed text. The test apparatus 300 may provide the generated request prompt to the large language model and acquire evaluation feedback related to fluency in response to the request prompt from the large language model. The test apparatus 300 may extract linguistic features based on the evaluation feedback and the transcribed text.

According to one embodiment, the test apparatus 300 may classify the user according to the degree of cognitive impairment based on the acoustic features and the linguistic features. For example, the test apparatus 300 may classify the user into a dementia group, a mild cognitive impairment group, or a normal group based on the acoustic features and the linguistic features.

According to one embodiment, the test apparatus 300 may generate a test opinion that summarizes the classification result of the degree of cognitive impairment and the evaluation feedback through the large language model. For example, the test apparatus 300 may provide a prompt including the classification result of the degree of cognitive impairment, the evaluation feedback, and a request for summary to the large language model through a designated communication channel. The test apparatus 300 may provide a test result (text) including the classification result and the evaluation feedback summarized by the large language model to the user.

According to one embodiment, the test apparatus 300 may provide the test result to the user terminal 100 through a designated communication channel. Accordingly, the test apparatus 300 according to one embodiment may convert the classification result expressed in numbers into text in a form recognizable to the user.

As described above, the system 10 for classifying cognitive impairment according to one embodiment may generate evaluation feedback related to fluency of a user using a large language model and may additionally use the evaluation feedback in addition to utterance voice of the user to classify the cognitive impairment of the user (examinee). Therefore, the test apparatus 300 according to the embodiment may provide cognitive impairment classification performance corresponding to a large language model even when built-in a small-scale artificial intelligence model.

FIG. 2 is a block diagram illustrating a configuration of a test apparatus according to an embodiment.

Referring to FIG. 2, the test apparatus 300 according to one embodiment may include at least one of a processor 310, a memory 330, an input interface apparatus 350, an output interface apparatus 360, and a storage apparatus 340 that communicate through a bus 370. The test apparatus 300 may also include a communication apparatus 320 coupled to a network. In one embodiment, the test apparatus 300 may not include some of the components or further include additional components. In addition, some of the components of the test apparatus 300 may be combined to form a single component, but may perform the same functions of the components before the combination.

The communication module 320 may support establishment of a communication channel or a wireless communication channel between the test apparatus 300 and other apparatuses (e.g., the user terminal 100 and the server apparatus 200), and performance of communication through the established communication channel. The communication channels may include, for example, at least one communication channel among a local area network (LAN), fiber to the home (FTTH), a digital subscriber line (xDSL), wireless broadband (WiBro), a wireless LAN, Wi-Fi, Bluetooth, Zigbee, Wi-Fi Direct (WFD), ultra-wideband (UWB), infrared communication (IrDA; Infrared Data Association), Bluetooth Low Energy (BLE), near field communication (NFC), 3G, 4G, or 5G.

The memory 330 may include various types of volatile memories or non-volatile memories. For example, the memory 330 may include a read only memory (ROM) and a random-access memory (RAM). In one embodiment, the memory 330 may be located inside or outside the processor 310, and the memory 330 may be connected to the processor 310 through various known means. The memory 330 may store various types of data used by at least one component (e.g., the processor 310) of the test apparatus 300. The data may include, for example, software and input data or output data regarding instructions related thereto. For example, the memory 330 may store at least one instruction related to classification of cognitive impairment groups using an artificial intelligence model 335.

The processor 310 may control at least one other component (e.g., a hardware component or a software component) of the test apparatus 300 and may perform various data processing processes or calculations. The processor 310 may include, for example, a central processing unit (CPU), a graphics processing unit (GPU), a microprocessor, an application processor, an application specific integrated circuit (ASIC), or a field programmable gate array (FPGA), and may have a plurality of cores.

According to one embodiment, the processor 310 may execute at least one instruction, to extract acoustic features based on utterance voice of a user and extract linguistic features based on transcribed text corresponding to the utterance voice, generate a request prompt related to a fluency evaluation based on the transcribed text and input the request prompt into a large language model and acquire evaluation feedback from the large language model, and classify the user according to the degree of cognitive impairment based on the evaluation feedback, the acoustic features, and the linguistic features.

The processor 310 may execute the at least one instruction, to request training data of the artificial intelligence model in relation to images, utterance voice, and transcribed text generated from cognitively impaired patients to the large language model, and construct the artificial intelligence model 335 based on the training data. For example, the processor 310 may request training data of the artificial intelligence model 335 in relation to images, utterance voice, and transcribed text generated from cognitively impaired patients to the large language model, receive the train data from the large language model in response to the request, and construct the artificial intelligence model 335 based on the training data.

The processor 310 may execute the at least one instruction to acquire utterance voice from the user terminal 100 through the communication module 320 and provide the test opinion to the user terminal 100 through the communication module 320.

The processor 310 may execute the at least one instruction to generate the request prompt including the transcribed text and at least one evaluation criterion text among an explanatory phrase related to the transcribed text, a score range and a score unit of the fluency evaluation, and example transcribed text rated with a plurality of scores within the score range.

The processor 310 may execute the at least one instruction to generate the prompt that may allow acquisition of the fluency or clarity evaluation feedback related to at least one of an inclusion of key elements, a repetition, and contextually unclear phrases in the transcribed text.

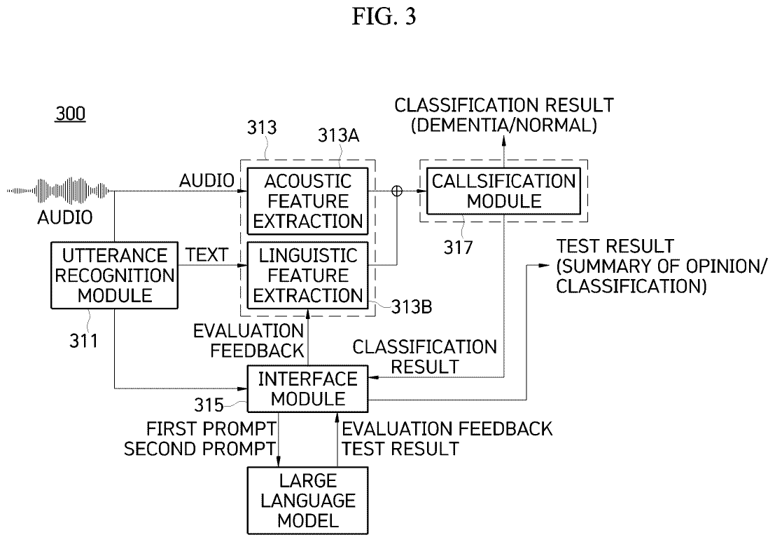

FIG. 3 is a block diagram illustrating a detailed configuration of a test apparatus according to an embodiment.

Referring to FIG. 3, the test apparatus 300 according to one embodiment may include a utterance recognition module 311, an extraction module 313, an interface module 315, and a classification module 317. In one embodiment, the utterance recognition module 311, the extraction module 313, the interface module 315, and the classification module 317 may be a software module or a hardware module included in the processor 310 or executed by the processor 310.

According to one embodiment, the utterance recognition module 311 may acquire utterance voice of a user from the communication module 320 and convert the utterance voice into text, thereby generating transcribed text. The utterance voice may include utterance of a user (an examinee) who describes an image (a painting or a photo). The utterance recognition module 311 is a machine learning model that converts utterance into text, for example, a Wisper ASR system.

According to one embodiment, the extraction module 313 may include a first extraction unit 313A and a second extraction unit 313B.

The first extraction unit 313A may acquire utterance voice of a user and extract acoustic features from the utterance voice. The acoustic features may include, for example, fluency-related features including at least one of pitch, a range of intensity, and an utterance pause duration of the user. The first extraction unit 313A is an artificial intelligence model capable of extracting an acoustic feature vector from a utterance input, and may be, for example, a Wav2Vec 2.0 model.

The second extraction unit 313B may extract linguistic features based on transcribed text and evaluation feedback (text). The evaluation feedback may be acquired from the large language model through the interface module 315, for example. The linguistic features may include linguistic features required for identifying fluency (and/or clarity), such as at least one of phonemic paraphasia, semantic paraphasia, inversion, interprojection, modification, onset time of subsequent utterances, repetition (e.g. sound repetition, syllable repetition, and phrase repetition), whether key phrases are included, and incomplete and unclear expressions. The second extraction unit 313B may use, for example, a BERT model.

According to one embodiment, the interface module 315 may generate a first prompt related to a fluency evaluation feedback request based on transcribed text. For example, the interface module 315 may generate a first prompt including the transcribed text and at least one evaluation criterion text among an explanatory phrase related to transcribed text, a score range and a score unit (e.g., an integer scale within 1 to 10) of the fluency evaluation, and example transcribed text rated with a plurality of scores (e.g., the lowest score and the highest score) within the score range. The explanatory phrase may include, for example, the name of an image described by the user, key elements within the image, and a detailed explanation about the image. When the image is an image published on the web, the detailed explanation of the image may be omitted. In various embodiments, the first prompt may include the image (an image described by the user) according to the transcribed text in addition to or instead of the explanatory phrase. The example transcribed text include, for example, first transcribed text rated with the lowest score by the MMSE and second transcribed text rated with the highest score by the MMSE, and the example transcribed text may be provided in association with scores.

According to one embodiment, the interface module 315 may input the generated first prompt into the large language model and acquire evaluation feedback in response to the first prompt from the large language model. The evaluation feedback may include fluency (or clarity) feedback related to at least one of a use rate of key elements in the image, consistency of description, repeated use of unnecessary terms, and use of conceptually unclear phrases.

According to one embodiment, the classification module 317 may acquire the acoustic features and the linguistic features as input from the extraction module 313, and classify the degree of cognitive impairment of the user based on the acoustic features and the linguistic features. For example, the classification module 317 may classify the user into one of a dementia group, a mild cognitive impairment group, or a normal group based on the linguistic features and the acoustic features which correspond to the transcribed text and the evaluation feedback. As another example, the classification module 317 may classify the user into one of the dementia group, the mild cognitive impairment group, and the normal group based on a similarity between the user's features (the linguistic features and the acoustic features) and reference features (e.g., linguistic features and acoustic features of each of the dementia group, the mild cognitive impairment group, and the normal group). In one embodiment, the classification module 317 may be a classifier including, for example, a transformer block and a linear radar. The classification module 317 may be constructed by learning classification of the degree of cognitive impairment based on acoustic features and linguistic features.

Meanwhile, when the classification by the classification module 317 is completed, the interface module 315 may generate a second prompt related to a request for a summary of the classification result and the evaluation feedback. The second prompt may include the classification result, the evaluation feedback, and a request for summary thereof. The classification result may include a numerical value (e.g., a similarity value) that may be interpreted by artificial intelligence.

The interface module 315 may transmit the second prompt to the large language model and acquire a test result in response to the second prompt from the large language model. The test result is text summarizing the classification result and the evaluation feedback and may be configured for the user to easily identify the degree of the cognitive impairment. To this end, the interface module 315 may be configured to generate the second prompt such that the test result may be acquired in a desired form through learning. The test result may include, for example, a cognitive impairment group to which the user belongs, symptoms identified in the transcribed text, characteristics of symptoms of the cognitive impairment group to which the user belongs, and overall comments from the result of the test. The generated test result, for example, may indicate that the user is classified into the dementia group, that there are symptoms identified in the transcribed text, including difficulty conveying a clear and consistent narrative about an image, repeated statements, and contextually nonsensical expressions, and include a summary of overall comments thereof. Additionally, the test result may include a description of characteristics of dementia patients and symptoms that may appear later.

According to one embodiment, the interface module 315 may provide the test result (or the classification result and the test result) to the user terminal 100 through the communication module 320.

In the above-described embodiment, the interface module 315 may learn to generate the first prompt that may acquire evaluation feedback based on transcribed text. The interface module 315 may learn to generate the second prompt that may acquire a test result based on the evaluation feedback and the classification result.

According to various embodiments, the training data of the interface module 315 and the classification module 317 may be obtained using the large language model. For example, the test apparatus 300 may acquire utterance voice of a dementia patient describing a first image, and obtain training data similar to the acquired utterance voice of the cognitively impaired patient through the large language model. For example, the interface module 315 may generate transcribed text for training from utterance voice of a dementia patient. And the interface module 315 may input the generated transcribed text for training and a third prompt requesting generation of N pieces of transcription data for training similar to the generated transcribed text for training into the large language model. The interface module 315 may obtain N pieces of transcription data for training in response to the third prompt from the large language model. The classification module 317 may learn to classify the degree of cognitive impairment based on acoustic features and linguistic features based on the N pieces of transcription data for training.

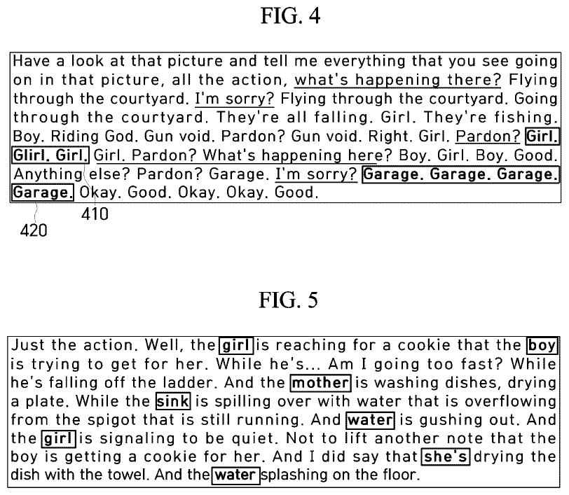

FIGS. 4 and 5 are examples of transcribed text generated by presenting the “cookie theft” photo as part of the Boston Diagnostic Aphasia test, which is frequently used for dementia patients, recording utterance voice of users describing the photo, and transcribing the utterance.

As shown in the transcribed text in FIG. 4, it can be found that a user in the dementia group repeatedly utters certain words (410) or that an assistant frequently intervenes in the description process due to lack of expressiveness (420).

On the other hand, as shown in the transcribed text in FIG. 5, it can be found that a user in the normal group fluently describes various items, such as girl, boy, mother, sink, and water.

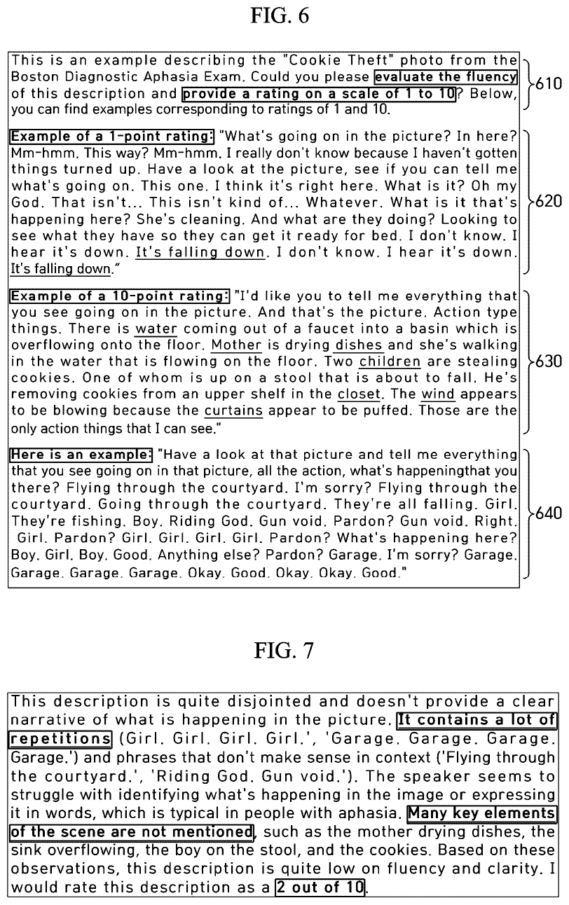

FIG. 6 is an example of the first prompt according to one embodiment.

Referring to FIG. 6, the first prompt may include an explanatory phrase indicating that target transcribed text describes the cookie theft photo, a request to evaluate the fluency regarding how fluently the user describes the photo, and a request to rate a score between 1 and 10 in a first area 610. The first prompt may include example transcribed text with a fluency score of 1 in a second area 620 and example transcribed text with a fluency score of 10 in a third area 630. For example, the transcribed text examples with fluency scores of 1 and 10 may be determined to be one user's utterance (or utterance-based transcribed text) with the lowest MMSE scale and another user's utterance (or utterance-based transcribed text) with the highest MMSE scale among previously stored utterance data of other users. Additionally, the first prompt may include transcribed text to be subject to evaluation corresponding to utterance voice of a user (an examinee) in a fourth area 640.

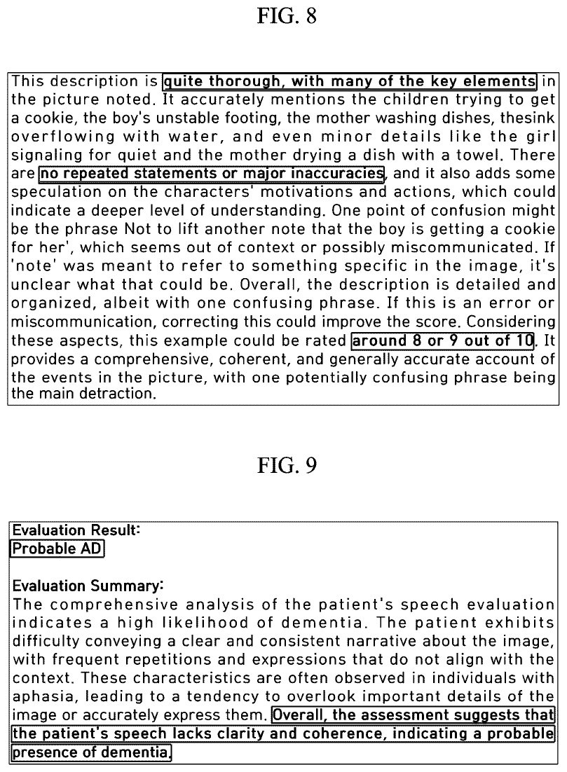

FIG. 7 is an example of fluency evaluation feedback based on a large language model for an utterance of a user in the dementia group according to an embodiment, and FIG. 8 is an example of fluency evaluation feedback based on a large language model for an utterance of a user in the normal group according to an embodiment.

Referring to FIG. 7, the large language model provides evaluation feedback rated with a score of 2 out of 10 points for the utterance of the user in the dementia group, emphasizing that the utterance is highly inconsistent, has much repetition and many contextually incomprehensive phrases, and has many key elements of the scene omitted.

Referring to FIG. 8, the large language model provides evaluation feedback rated on a scale of 8 to 9 points for the utterance of the user in the normal group, indicating that many key elements are well expressed, and there are almost no repeated statements or major inaccuracies.

The evaluation feedback such as that shown in FIGS. 7 and 8 may be input into the second extraction unit 313B and extracted as part of linguistic features through the second extraction unit 313B. Thereafter, the classification module 317 may further use the linguistic features of the evaluation feedback in addition to utterance of a user to classify the degree of cognitive impairment of the user into a normal group, a mild cognitive impairment group, or a dementia group. The evaluation feedback such as that shown in FIGS. 7 and 8 may be provided to the interface module 315 together with a classification result of the user by the classification module 317. Thereafter, the interface module 315 may input a second prompt requesting a text summary based on the evaluation feedback and the classification result of the degree of cognitive impairment into the large language model. As a result, the interface module 315 may acquire a test result from the large language model.

FIG. 9 is an example of a test result according to one embodiment.

Referring to FIG. 9, the test opinion may indicate that the user is classified into the dementia group, that there are symptoms identified in the transcribed text, including difficulty in conveying a clear and consistent narrative about an image and repeated statements and contextually nonsensical expressions, and include characteristics and potential symptoms of dementia patients and a summary of overall comments.

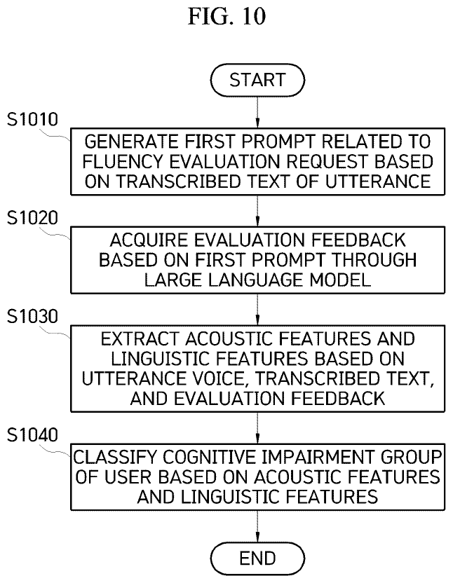

FIG. 10 is a flowchart showing a method of classifying cognitive impairment according to one embodiment.

Referring to FIG. 10, in operation 1010, the test apparatus 300 may generate a first prompt related to a fluency evaluation request based on transcribed text corresponding to utterance voice of a user.

In operation 1020, the test apparatus 300 may acquire evaluation feedback related to fluency in response to the first prompt through a large language model.

In operation 1030, the test apparatus 300 may extract acoustic features and linguistic features based on the utterance voice, the transcribed text, and the evaluation feedback.

In operation 1040, the test apparatus 300 may classify a cognitive impairment group to which the user belongs based on the acoustic features and the linguistic features.

The various embodiments of the disclosure and terminology used herein are not intended to limit the technical features of the disclosure to the specific embodiments, but rather should be understood to cover all modifications, equivalents, and alternatives falling within the spirit and scope of the disclosure. Like numbers refer to like elements throughout the description of the drawings. The singular forms preceded by “a,” “an,” and “the” corresponding to an item are intended to include the plural forms as well unless the context clearly indicates otherwise. In the disclosure, a phrase such as “A or B,” “at least one of A and B,” “at least one of A or B,” “A, B or C,” “at least one of A, B and C,” and “at least one of A, B, or C” may include any one of the items listed together in the corresponding phrase, or any possible combination thereof. Terms such as “first,” “second,” etc. are used to distinguish one element from another and do not modify the elements in other aspects (e.g., importance or sequence). When one (e.g., a first) element is referred to as being “coupled” or “connected” to another (e.g., a second) element with or without the term “functionally” or “communicatively,” it means that the one element is connected to the other element directly (e.g., by wire), wirelessly, or via a third element.

As used herein, the term “module” may include units implemented in hardware, software, or firmware, and may be interchangeably used with terms such as “logic,” “logic block,” “component,” or “circuit.” The module may be an integrally configured component or a minimum unit or part of the integrally configured component that performs one or more functions. For example, according to one embodiment, the module may be implemented in the form of an application-specific integrated circuit (ASIC).

The various embodiments of the present disclosure may be realized by software (e.g., a program) including one or more instructions stored in a storage medium (e.g., the memory 530, such as an internal memory or external memory,) that can be read by a machine (e.g., the apparatus 500 for image recognition). For example, a processor (e.g., the processor 510) of the machine (e.g., the apparatus for image recognition) may invoke and execute at least one instruction among the stored one or more instructions from the storage medium. Accordingly, the machine operates to perform at least one function in accordance with the invoked at least one command. The one or more instructions may include codes generated by a compiler or codes executable by an interpreter. The machine-readable storage medium may be provided in the form of a non-transitory storage medium. Here, when a storage medium is referred to as “non-transitory,” it can be understood that the storage medium is tangible and does not include a signal (for example, electromagnetic waves), but rather that data is semi-permanently or temporarily stored in the storage medium.

According to one embodiment, the methods according to the various embodiments disclosed herein may be provided in a computer program product. The computer program product may be traded between a seller and a buyer as a product. The computer program product may be distributed in the form of a machine-readable storage medium (e.g., a compact disc read only memory (CD-ROM)), or may be distributed directly between two user devices (e.g., smartphones) through an application store (e.g., Play Store™), or online (e.g., downloaded or uploaded). In the case of online distribution, at least a portion of the computer program product may be stored at least semi-permanently or may be temporarily generated in a machine-readable storage medium, such as a memory of a server of a manufacturer, a server of an application store, or a relay server.

Components according to various embodiments of the disclosure 0 may be implemented in the form of software or hardware, such as a digital signal processor (DSP), a field programmable gate array (FPGA) or an application specific integrated circuit (ASIC), and may perform predetermined functions. The “elements” are not limited to meaning software or hardware. Each of the elements may be configured to be stored in a storage medium capable of being addressed and configured to execute one or more processors. For example, the elements may include elements such as software elements, object-oriented software elements, class elements, and task elements, processes, functions, attributes, procedures, subroutines, segments of a program code, drivers, firmware, microcode, circuits, data, databases, data structures, tables, arrays, and variables.

According to the various embodiments, each of the above-described elements (e.g., a module or a program) may include a singular entity or a plurality of entities. According to various embodiments, one or more of the above described elements or operations may be omitted, or one or more other elements or operations may be added. Alternatively or additionally, a plurality of elements (e.g., modules or programs) may be integrated into one element. In this case, the integrated element may perform one or more functions of each of the plurality of elements in a manner the same as or similar to that performed by the corresponding element of the plurality of components before the integration. According to various embodiments, operations performed by a module, program, or other elements may be executed sequentially, in parallel, repeatedly, or heuristically, or one or more of the operations may be executed in a different order, or omitted, or one or more other operations may be added.

As is apparent from the above, according to various embodiments, cognitive impairment can be tested based on evaluation feedback on utterance voice by a user and features of the utterance voice. In addition, various effects that can be directly or indirectly identified through this disclosure can be provided.

What is claimed is:

1. An electronic apparatus comprising:
an interface module configured to generate a first prompt related to a fluency evaluation request based on utterance voice of a user, and acquire evaluation feedback related to fluency based on the first prompt through a large language model;
a first extraction unit configured to extract acoustic features based on the utterance voice;
a second extraction unit configured to extract linguistic features based on transcribed text corresponding to the utterance voice and the evaluation feedback; and
a classification module configured to classify a cognitive impairment group to which the user belongs based on the acoustic features and the linguistic features.

2. The electronic apparatus of claim 1, wherein the utterance voice includes a voice of the user that describes a painting or photo.

3. The electronic apparatus of claim 1, wherein the interface module is configured to generate the first prompt including the transcribed text and at least one evaluation criterion text among an explanatory phrase related to the transcribed text, a score range and a score unit of the fluency evaluation, and example transcribed text associated with a plurality of scores within the score range.

4. The electronic apparatus of claim 3, wherein the plurality of scores include a lowest score and a highest score within the score range.

5. The electronic apparatus of claim 1, wherein the interface module is configured to generate the first prompt allowing acquisition of fluency evaluation feedback related to at least one of inclusion of key elements, consistency, term repetition, and wording accuracy in the transcribed text.

6. The electronic apparatus of claim 1, wherein the classification module is configured to classify the user into one of a dementia group, a mild cognitive impairment group, or a normal group based on the linguistic features and the acoustic features.

7. The electronic apparatus of claim 1, wherein the interface module is configured to generate a test opinion that summarizes a result of the classification on the cognitive impairment and the evaluation feedback through the large language model.

8. A method of classifying cognitive impairment, the method comprising:
generating a first prompt related to a fluency evaluation request based on transcribed text corresponding to utterance voice of a user;
acquiring evaluation feedback related to fluency in response to the first prompt through a large language model;
extracting acoustic features and linguistic features based on the utterance voice, the transcribed text, and the evaluation feedback; and
classifying a cognitive impairment group to which the user belongs based on the acoustic features and the linguistic features.

9. The method of claim 8, wherein the utterance voice includes voice of the user that describes a painting or photo.

10. The method of claim 8, wherein the generating of the first prompt includes:
generating the first prompt including the transcribed text and at least one evaluation criterion text among explanatory phrase related to the transcribed text, a score range and a score unit of the fluency evaluation, and example transcribed text rated with a plurality of scores within the score range.

11. The method of claim 10, wherein the plurality of scores include a lowest score and a highest score within the score range.

12. The method of claim 8, wherein the generating of the first prompt includes:
generating the first prompt that allows acquisition of fluency evaluation feedback related to at least one of inclusion of key elements, consistency, term repetition, and wording accuracy in the transcribed text.

13. The method of claim 8, wherein the classifying of the cognitive impairment group includes:
classifying the user into one of a dementia group, a mild cognitive impairment group, or a normal group based on the linguistic features and the acoustic features according to the transcribed text and the evaluation feedback.

14. The method of claim 8, further comprising:
summarizing a result of the classification on the cognitive impairment and the evaluation feedback through the large language model to generate a test opinion.

15. An electronic apparatus comprising:
a memory in which at least one instruction related to an artificial intelligence model is stored; and
a processor functionally connected to the memory, the processor executing the at least one instruction to:
generate a first prompt related to a fluency evaluation request based on transcribed text corresponding to utterance voice of a user;
input the first prompt to a large language model, and acquire evaluation feedback from the large language model;
extract acoustic features and linguistic features based on the utterance voice, the evaluation feedback, and the transcribed text; and
classify a cognitive impairment group to which the user belongs based on the acoustic features and the linguistic features.

16. The electronic apparatus of claim 15, wherein the processor executes the at least one instruction to:
request training data of the artificial intelligence model in relation to an image, utterance voice, and transcribed text of a cognitively impaired patient from the large language model; and
construct the artificial intelligence model based on the training data.

17. The electronic apparatus of claim 15, further comprising a communication module,
wherein the processor executes the at least one instruction to:
acquire the utterance voice from an external electronic apparatus through the communication module and provide the test opinion to the external electronic apparatus through the communication module.

18. The electronic apparatus of claim 15, wherein the processor executes the at least one instruction to:
generate the first prompt including the transcribed text and at least one evaluation criterion text among an explanatory phrase related to the transcribed text, a score range and a score unit of the fluency evaluation, and example transcribed text rated with a plurality of scores within the score range.

19. The electronic apparatus of claim 15, wherein the processor executes the at least one instruction to:
generate the first prompt allowing acquisition of fluency evaluation feedback related to at least one of inclusion of key elements, consistency, term repetition, and wording accuracy in the transcribed text.

20. The electronic apparatus of claim 15, wherein the processor executes the at least one instruction to:
summarize a result of the classification on the cognitive impairment and the evaluation feedback through the large language model to generate a test opinion.
